(19) United States
(12) Patent Application Publication
(10) Pub. No.: US 2026/0248840 A1
(43) Pub. Date: Aug. 27, 2026

(54) Chondroitin- and Collagen Peptide-Based Methods for Slowing Down Aging and Extending Lifespan

(71) Applicant: AgeLess Sciences, Inc., New York, NY (US)

(72) Inventors: Kris Verburgh, Willebroek (BE); Chris Mirabile, West Palm Beach, FL (US)

(21) Appl. No.: 18/862,225

(22) PCT Filed: May 4, 2023
(86) PCT No.: PCT/US2023/020961

§ 371 Date: Nov. 1, 2024

Related U.S. Application Data
(60) Provisional application No. 63/339,094, filed on May 6, 2022.

(51) Int. Cl. A61K 31/726; A61K 8/65; A61K 8/73; A61K 9/00; A61K 31/132; A61K 31/194; A61K 31/352; A61K 31/4415; A61K 31/51; A61K 31/525; A61K 31/685; A61K 31/7016; A61K 31/714; A61K 36/185; A61K 38/01; A61K 38/39; A61Q 19/08

(52) U.S. Cl. CPC A61K 31/726; A61K 8/65; A61K 8/735; A61K 9/0002; A61K 9/0095; A61K 31/132; A61K 31/194; A61K 31/352; A61K 31/4415; A61K 31/51; A61K 31/525; A61K 31/685; A61K 31/7016; A61K 31/714; A61K 36/185; A61K 38/014; A61K 38/39; A61Q 19/08; A61K 2800/88

(57) ABSTRACT

This invention provides a composition comprising (i) chondroitin and/or collagen peptide and (ii) at least one agent selected from the group consisting of butyrate, a butyrate equivalent, trehalose, a trehalose equivalent, spermidine, and a spermidine equivalent. This invention also provides related pharmaceutical compositions, methods for ameliorating aging hallmarks in a subject, and articles of manufacture.

This application claims the benefit of U.S. Provisional Application No. 63/339,094, filed May 6, 2022, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to compositions and methods for ameliorating one or more aging hallmarks in a subject.

### BACKGROUND OF THE INVENTION

Aging, in the physical sense, is characterized by the accumulation of molecular and cellular damage. This damage leads to a deterioration of physiological function and proper homeostasis, and increases the risk of frailty, infirmity, disability, aging-related diseases, and eventually death.

For a long time, one of the main causes of aging was considered to be oxidative damage, as promoted and promulgated by Denham Harman's free radical theory of aging (1). According to this dated theory, antioxidants have the ability to neutralize free radicals, thus slowing the aging process. Yet, research shows that most antioxidants do not slow down aging, and could even shorten lifespan or undo the beneficial effects of exercise (2-6). However, in the last 15 years, a better understanding of the aging process has emerged, leading to an understanding of various mechanisms and causes of aging. These include, for example, epigenetic dysregulation, epitranscriptomic dysregulation, a decline in proteinases (proteotoxicity), mitochondrial dysfunction, genomic damage, telomere shortening and attrition, transcriptomic dysregulation, lysosomal dysfunction and crosslinking contributing to the formation of senescent cells, a decline in stem cell function and numbers, changes of the extracellular milieu (e.g., a change to a pro-inflammatory milieu), and changes in intercellular communication. These changes contribute to the aging phenotype and an increased risk and an occurrence of aging-related diseases like heart disease, neurodegenerative diseases (e.g., Alzheimer's disease or vascular dementia), osteoporosis, osteoarthritis, macular degeneration, kidney dysfunction, and others.

There remains an unmet need for superior formulations to ameliorate these hallmarks of aging.

### SUMMARY OF THE INVENTION

This invention provides a composition comprising (i) chondroitin and/or collagen peptide and (ii) at least one agent selected from the group consisting of butyrate, a butyrate equivalent, trehalose, a trehalose equivalent, spermidine, and a spermidine equivalent.

This invention also provides a pharmaceutical composition comprising (i) the present composition and (ii) a pharmaceutically acceptable carrier.

This invention further provides a method for ameliorating at least one aging hallmark in a subject comprising administering to the subject the present composition, wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, transcriptomic dysregulation, crosslinking, and altered intercellular communication.

Finally, this invention provides an article of manufacture comprising, in separate compartments, (i) the present composition, wherein the composition does not comprise butyrate or a butyrate equivalent, and (ii) butyrate and/or a butyrate equivalent. This invention also provides an article of manufacture comprising the present butyrate- or butyrate equivalent-comprising composition, wherein (i) the butyrate and/or butyrate equivalent and (ii) other components of the composition are in separate compartments.

### DETAILED DESCRIPTION OF THE INVENTION

This invention provides compositions and methods for ameliorating an array of aging hallmarks in a subject.

### Definitions

In this application, certain terms are used which shall have the meanings set forth as follows.

As used herein, “administer”, with respect to an agent, means to deliver the agent to a subject's body via any known method. Specific modes of administration include, without limitation, oral, sublingual, parenteral, intraperitoneal, intravenous, intra-arterial, transdermal, intramuscular, rectal, transbuccal, intranasal, liposomal, via inhalation, vaginal, intraocular, local (e.g., via catheter or stent), subcutaneous, intrathecal, or intra-adiposal administration. Preferably, the present compositions are administered orally.

In addition, in this invention, the various compositions can be formulated using one or more routinely used pharmaceutically acceptable carriers. Such carriers are well known to those skilled in the art. For example, oral delivery systems include, for example, tablets, capsules, and powders (such as powders that can be added to water to form a drinkable formulation). These can contain excipients such as binders (e.g., hydroxypropylmethyl-cellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). In one embodiment, the oral delivery system is a powder and comprises one or more of xylitol, erythritol, malic acid, flavoring, silicon dioxide, calcium silicate, and rebaudioside A.

As used herein, the term “aging hallmark” (used synonymously with “age-related hallmark” and “hallmark related to aging”) includes, without limitation, any of genomic instability, telomere attrition, an epigenetic alteration, loss of proteostasis, deregulated nutrient sensing, mitochondrial dysfunction, cellular senescence, stem cell exhaustion, transcriptomic dysregulation, crosslinking, and altered intercellular communication. These hallmarks are known, and reviewed in depth by C. López Otín, et al. (7).

As used herein, the term “ameliorate”, when used in reference to an aging hallmark, includes, without limitation, (i) slowing, stopping, reversing, or preventing the hallmark's progression, (ii) slowing, stopping, reversing, or preventing the progression of the hallmark's symptoms, (iii) preventing or reducing the likelihood of the hallmark's recurrence, and/or (iv) preventing or reducing the likelihood that the hallmark's symptoms will recur. In one embodiment, treating a subject afflicted with an aging hallmark means (i) reversing the hallmark's progression, ideally to the point of eliminating the hallmark, and/or (ii) reversing the progression of the hallmark's symptoms, ideally to the point of eliminating the symptoms.

As used herein, “butyrate equivalent” includes, without limitation, a histone deacetylase (HDAC) inhibitor other than butyrate. These include, for example, butyric acid, butyrate salts (e.g., calcium butyrate, sodium butyrate, potassium butyrate, ammonium butyrate, and lithium butyrate), tributyrate, tributyrin, butyrin, glycerin tri-butyrate, phenylbutyrate, methylbutyrate, sodium phenylbutyrate, glycerol esters of butyric acid, amide derivatives of butyric acid, β-hydroxybutyrate (BHB), β-hydroxybutyric acid, β-hydroxybutyrate (BHB)-derivatives, β-hydroxybutyrate salts (e.g., potassium β-hydroxybutyrate, sodium β-hydroxybutyrate, calcium β-hydroxybutyrate, and magnesium β-hydroxybutyrate), β-hydroxybutyrate-based ketones, β-hydroxybutyrate-based ketone esters, β-hydroxybutyrate-based esters, β-hydroxybutyrate esters, β-hydroxybutyrate butanediol, β-hydroxybutyrate glycerol, R-1,3-butanediol-R-3-hydroxybutyrate, butanediol, R-1,3-butandediol, R-3-hydroxybutyrate, bis-hexanoyl-1,3-butanediol, β-ketopentanoate, β-hydroxypentanoate, valeric acid, valerian-extract containing valeric acid, valerian root, valproate, valproic acid, an alkyl carboxylic acid, an alkyl carboxylic acid-derivative, precursors of butyrate and butyric acid (e.g., fibers such as inulin, fructo-oligosaccharides (FOS), resistant starch, Hi-maize, potato starch, arabinoxylan, and beta-glucans), and combinations thereof. Butyrate equivalent also includes, without limitation, another alkane carboxylic acid or alkane carboxylic acid derivative (e.g., propionic acid or other short chain fatty acids such as formic acid, acetic acid, isovaleric acid, valeric acid, isocaproic acid, caproic acid, caprylic acid, lactic acid, succinic acid, pyruvic acid, and combinations thereof).

As used herein, “collagen peptides” include, without limitation, collagen, collagen hydrolysate, collagen tripeptides, collagen dipeptides, collagen monopeptides, derivatives, and combinations thereof. The term “collagen peptide”, as used herein, can mean, for example, a single peptide or type of peptide, as well as a mixture of two or more types of such peptide.

As used herein, a “phospholipid” includes, without limitation, phosphatidylcholine, phosphatidylserine, phosphatidylethanolamine, phosphatidic acid, a phosphoinositide, phosphatidylinositol, phosphatidylinositol phosphate (PIP), phosphatidylinositol bisphosphate (PIP2), phosphatidylinositol triphosphate (PIP3), a phosphatidyl ester, a glycerophospholipid, a plasmalogen, an ether lipid, a phosphosphingolipid, and derivatives and combinations thereof.

As used herein, a “polyamine” includes, without limitation, spermidine, putrescine, spermine, norspermidine, acetyl spermidine, diacetyl spermine, methylated spermidine, a spermidine salt, and derivatives and combinations thereof. Sources of polyamines include, without limitation, wheat germ, wheat germ extract, soybean, soybean extract, other extracts high in spermidine or polyamine content, and spermidine-rich foods.

As used herein, a “proteostasis-supporting saccharide” includes, without limitation, trehalose, trehalose polysaccharides, mannitol, glucosamine, acetyl-glucosamine, galactosamine, mannosamine, and derivatives and combinations thereof.

As used herein, “spermidine equivalent” includes, without limitation, putrescine, spermine, norspermidine, acetyl spermidine, diacetyl spermine, methylated spermidine, methylated polyamines, spermidine salts, and other polyamines that help to protect cells from oxidative damage. Spermine and its equivalents are found, for example, in wheat germ, wheat germ extract, soybeans, and soybean extract.

As used herein, the term “subject” includes, without limitation, a mammal such as a human, a non-human primate, a dog, a cat, a horse, a sheep, a goat, a cow, a rabbit, a pig, a rat, and a rodent (such as a mouse). Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger.

As used herein, “trehalose equivalent” includes, without limitation, mannitol, glucosamine, acetyl-glucosamine, galactosamine, mannosamine, glycerol, erythritol, xylitol, and other saccharide-based molecules that help to protect cells against stress and/or to improve proteotoxicity.

### Embodiments of the Invention

This invention provides compositions and methods for ameliorating aging hallmarks. These compositions comprise certain combinations of agents that act in concert to address hallmarks of aging such as genomic instability, telomere attrition, epigenetic alterations, loss of proteostasis, deregulated nutrient sensing, mitochondrial dysfunction, cellular senescence, stem cell exhaustion, and altered intercellular communication. The invention also provides related articles of manufacture.

Specifically, this invention provides a composition comprising (i) chondroitin and/or collagen peptide and (ii) at least one agent selected from the group consisting of butyrate, a butyrate equivalent, trehalose, a trehalose equivalent, spermidine, and a spermidine equivalent.

In one embodiment of the present composition, the composition comprises (i) chondroitin and (ii) at least one agent selected from the group consisting of butyrate, a butyrate equivalent, trehalose, a trehalose equivalent, spermidine, and a spermidine equivalent. Preferably, the composition comprises chondroitin, butyrate, trehalose, and spermidine.

In another embodiment of the present composition, the composition comprises (i) collagen peptide and (ii) at least one agent selected from the group consisting of butyrate, a butyrate equivalent, trehalose, a trehalose equivalent, spermidine, and a spermidine equivalent. Preferably, the composition comprises collagen peptide, butyrate, trehalose, and spermidine.

In a further embodiment, the composition comprises (i) chondroitin, (ii) collagen peptide, and (iii) at least one agent selected from the group consisting of butyrate, a butyrate equivalent, trehalose, a trehalose equivalent, spermidine, and a spermidine equivalent. In a preferred embodiment, the composition comprises chondroitin, collagen peptide, butyrate, trehalose, and spermidine.

In one embodiment of the present composition, the trehalose equivalent is mannitol, glucosamine, acetyl-glucosamine, galactosamine, mannosamine, glycerol, erythritol, or xylitol. In another embodiment, the spermidine equivalent is putrescine, spermine, norspermidine, acetyl spermidine, diacetyl spermine, methylated spermidine, a methylated polyamine, or a spermidine salt. In a further embodiment, the butyrate equivalent is acetic acid.

In one example, the composition comprises 50 mg-6,000 mg of chondroitin, 50 mg-20,000 mg of collagen peptide, 50 mg-12,000 mg of butyrate, 0.1 g-50 g of trehalose, and 0.1 mg-30 mg of spermidine. In another example, the composition comprises 400 mg-1,200 mg of chondroitin, 1,000 mg-5,000 mg of collagen peptide, 400 mg-1,200 mg of butyrate, 1 g-20 g of trehalose, and 0.2 mg-20 mg of spermidine. In a preferred embodiment, the composition comprises 800 mg of chondroitin, 3,000 mg of collagen peptide, 800 mg of butyrate, 5 g of trehalose, and 2 mg of spermidine. In another preferred embodiment, the composition comprises 1,200 mg of chondroitin, 5,000 mg of collagen peptide, 1,100 mg of butyrate, 10 g of trehalose, and 2 mg of spermidine. Optionally, the present composition comprises one or more sweeteners such as, without limitation, erythritol, maltitol, mannitol, sorbitol, xylitol, isomalt, lactitol, tagatose, monk fruit, inuline, and stevia-derived substances.

In yet a further embodiment, the composition comprises (i) chondroitin, collagen peptide, butyrate, trehalose, and spermidine, and (ii) one or more agents selected from the group consisting of ascorbic acid or an ascorbic acid equivalent, alpha-ketoglutarate or an alpha-ketoglutarate equivalent, pomegranate or a pomegranate equivalent, a phospholipid, a B vitamin, and apigenin or an apigenin equivalent. In one embodiment of the present composition, the composition comprises chondroitin, collagen peptide, butyrate, trehalose, spermidine, ascorbic acid, and alpha ketoglutarate. In another embodiment, the composition comprises chondroitin, collagen peptide, butyrate, trehalose, spermidine, ascorbic acid, alpha ketoglutarate, and phosphatidylcholine. In a further embodiment, the composition comprises chondroitin, collagen peptide, butyrate, trehalose, spermidine, ascorbic acid, alpha ketoglutarate, phosphatidylcholine, and a B vitamin. In a further embodiment, the composition comprises chondroitin, collagen peptide, butyrate, trehalose, spermidine, ascorbic acid, alpha ketoglutarate, phosphatidylcholine, a B vitamin, and apigenin or an apigenin equivalent. In one example, the composition comprises one or more agents selected from the group consisting of 50 mg-2,000 mg of chondroitin, 200 mg-10,000 mg of collagen peptide, 100 mg-7,000 mg of butyrate, 0.2 g-20 g of trehalose, 0.1 mg-30 mg of spermidine, 10 to 2,000 mg of ascorbic acid, 100 mg-10,000 mg of alpha-ketoglutarate, 20 mg to 7,000 mg of pomegranate or pomegranate equivalent, 40 mg 5,000 mg of phosphatidylcholine, 5%-5,000% of the U.S. recommended daily allowance of B vitamins, and 50 mg of apigenin. In another example, the composition comprises 400 mg-1,200 mg of chondroitin, 1,000 mg-5,000 mg of collagen peptide, 400 mg-1,200 mg of butyrate, 1 g-20 g of trehalose, 0.2 mg-20 mg of spermidine, 500 mg of ascorbic acid, 500 mg-4,000 mg of alpha-ketoglutarate, 100 mg to 3,000 mg of pomegranate or pomegranate equivalent, 250 mg-2,400 mg of phosphatidylcholine, 100%-300% of the U.S. recommended daily allowance of B vitamins, and 50 mg of apigenin. Preferably, in this example, the composition comprises a daily dose of 1,200 mg of chondroitin, 5,000 mg of collagen peptide, 1,200 mg of butyrate, 10 g of trehalose, 2 mg of spermidine, 500 mg of ascorbic acid, 1,100 mg of alpha-ketoglutarate, 1,500 mg of pomegranate or pomegranate equivalent, 1,200 mg of phosphatidylcholine, 200% of the U.S. recommended daily allowance of B vitamins, and 50 mg of apigenin. Optionally, the present composition comprises one or more sweeteners such as, without limitation, erythritol, maltitol, mannitol, sorbitol, xylitol, isomalt, lactitol, tagatose, monk fruit, inuline, and stevia-derived substances.

Preferably, one or more of the agents in the present composition is formulated for delayed release and/or extended release. In one embodiment of the present composition, the chondroitin, collagen peptide, butyrate, trehalose, and spermidine are formulated for delayed release and/or extended release. In another embodiment of the present composition, butyrate and spermidine, and optionally trehalose, alpha-ketoglutarate, and ascorbic acid, are formulated for delayed release and/or extended release. In another embodiment of the present composition, each agent is formulated for delayed release and/or extended release. In still a further embodiment, the present composition comprises 1,200 mg of delayed release and/or extended release chondroitin, 5,000 mg of delayed release and/or extended release collagen peptide, 1,200 mg of delayed release and/or extended release butyrate, 10 g of delayed release and/or extended release trehalose, 2 mg of delayed release and/or extended release spermidine, 500 mg of delayed release and/or extended release ascorbic acid, 1,100 mg of delayed release and/or extended release alpha-ketoglutarate, 1,500 mg of delayed release and/or extended release pomegranate or pomegranate equivalent, 1,200 mg of delayed release and/or extended release phosphatidylcholine, 200% of the U.S. recommended daily allowance of B vitamins (in delayed release and/or extended release form), and 50 mg of delayed release and/or extended release apigenin. Optionally, the present composition comprises one or more sweeteners such as, without limitation, erythritol, maltitol, mannitol, sorbitol, xylitol, isomalt, lactitol, tagatose, monk fruit, inuline, or stevia-derived substances.

Each of the agents in the present composition is commercially available, for example, in oral dosage forms. The following are non-limiting examples.

Chondroitin is commercially available, for example, in 600 mg capsules. Chondroitin is available as the sulfate salt, but all other forms are envisioned in this invention. These include chains of acetylgalactosamine and glucuronic acid, mixtures of acetylgalactosamine and glucuronic acid, derivatives and combinations thereof. Collagen peptides are commercially available, for example, in powdered form for 5 g servings. Butyrate is commercially available, for example, in capsule form. Butyrate is available as the sodium salt, but all other salts of butyrate are envisioned in this invention. Also envisioned for use in this invention, in lieu of butyrate, are other histone deacetylase inhibitors. Phosphatidylcholine, also known as phosphatidyl choline, is commercially available, for example, as softgel capsules. Also envisioned for use in this invention, in lieu of phosphatidylcholine, is phosphatidylserine.

B vitamins include thiamin (vitamin B1), riboflavin (vitamin B2), niacin (vitamin B3), pantothenic acid, pyridoxine (vitamin B6), biotin (vitamin B7), folate and folic acid, and methylcobalamin (vitamin B12). They are commercially available, for example, individually and in complex “super B” forms such as softgels and tablets. Additional B vitamins and their derivatives include, without limitation, nicotinamide, nicotinamide riboside, nicotinamide riboside (NR), niacinamide, nicotinic acid, nicotinamide mononucleotide (NMN), nicotinic acid mononucleotide (NaMN), nicotinic acid riboside (NaR), nicotinamide adenine dinucleotide (NAD), NADH, nicotinamide riboside derivatives (or salts or prodrugs thereof or any molecule that can increase NAD+levels in cells and organisms), choline, carnitine, adenine (vitamin B4), pyridoxal, pyridoxamine (vitamin B6), inositol (vitamin B8), myo-inositol, adenosine monophosphate (AMP), methyl folate (vitamin B9), cobalamin, cyanocobalamin, choline, dimethylglycine (DMG) (vitamin B16), trimethylglycine or betaine (TMG), benfotiamine, phosphatidylcholine, and derivatives and combinations thereof.

Apigenin is commercially available, for example, in 35 mg capsules of liposomal apigenin. Alpha-ketoglutarate, also known as alpha-ketoglutaric acid, is commercially available, for example, in 1,000 mg capsules. Alpha-ketoglutarate is available as the calcium salt, but all other forms of alpha-ketoglutarate are envisioned in this invention. They include, for example, potassium alpha-ketoglutarate, sodium alpha-ketoglutarate, other salts of alpha-ketoglutarate, amino acid forms of alpha-ketoglutarate (e.g., arginine-alpha-ketoglutarate, alpha-ketoglutaric acid, and derivatives and combinations thereof).

Ascorbic acid, also known as vitamin C and ascorbate, is commercially available, for example, in 1,000 mg tablets. Also envisioned for use in this invention, in lieu of ascorbic acid, is an ascorbic acid derivative. These derivatives include, without limitation, ascorbyl palmitate, tetrahexyldecyl ascorbate, sodium ascorbyl phosphate, magnesium ascorbyl phosphate, sodium ascorbate, calcium ascorbate, ascorbic acid polypeptide, ascorbic acid peptide, ascorbyl glucosamine, ascorbyl glucoside, tetrahexyldecyl ascorbate, and combinations thereof.

Spermidine is commercially available, for example, in 400 mg capsules of wheat germ extract containing 0.5 mg of spermidine. Trehalose is commercially available in powder form.

Pomegranate is of course widely available, as are its component active ingredients (i.e., “pomegranate equivalents”). Pomegranate equivalents include, without limitation, pomegranate extract, ellagic acid, urolithins, urolithin, urolithin A, urolithin B, urolithin C, urolithin D, ellagic acid metabolites, ellagitannins, urolithin glucuronides, ursolic acid, maresin, and foods high in ellagic acid (e.g., berries, black raspberry, blackberry, cloudberry, strawberry, red raspberry, cranberries, acai, apple, grapes, walnuts, chestnuts, pecan, and arctic blackberry).

Additional embodiments of the present composition are set forth below in the Examples section.

This invention also provides a pharmaceutical composition comprising (i) the present composition and (ii) a pharmaceutically acceptable carrier.

In a preferred embodiment, the present composition is in a form selected from the group consisting of a food, a powdered drink mix (to be mixed with water and orally consumed), a nutritional supplement, a nutraceutical, and a skincare product. Preferably, each of these forms is self-administered daily.

This invention further provides a method for ameliorating at least one aging hallmark in a subject comprising administering to the subject (preferably once daily) the present composition, wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, transcriptomic dysregulation, crosslinking, and altered intercellular communication.

In a preferred embodiment, the present method ameliorates genomic instability, telomere attrition, one or more epigenetic alterations, loss of proteostasis, deregulated nutrient sensing, mitochondrial dysfunction, cellular senescence, stem cell exhaustion, transcriptomic dysregulation, crosslinking, and altered intercellular communication.

In one embodiment, the present method ameliorates genomic instability. In another embodiment, the present method ameliorates telomere attrition. In another embodiment, the present method ameliorates one or more epigenetic alterations. In another embodiment, the present method ameliorates loss of proteostasis. In another embodiment, the present method ameliorates deregulated nutrient sensing. In another embodiment, the present method ameliorates mitochondrial dysfunction. In another embodiment, the present method ameliorates cellular senescence. In another embodiment, the present method ameliorates stem cell exhaustion. In another embodiment, the present method ameliorates transcriptomic dysregulation. In another embodiment, the present method ameliorates crosslinking. In another embodiment, the present method ameliorates altered intercellular communication.

In a further embodiment of the present method, the method ameliorates at least two aging hallmarks in the subject. In another embodiment, the present method ameliorates at least three aging hallmarks in the subject. In another embodiment, the present method ameliorates at least four aging hallmarks in the subject. In another embodiment, the present method ameliorates at least five aging hallmarks in the subject. In another embodiment, the present method ameliorates at least six aging hallmarks in the subject. In another embodiment, the present method ameliorates at least seven aging hallmarks in the subject. In another embodiment, the present method ameliorates at least eight aging hallmarks in the subject. In another embodiment, the present method ameliorates at least nine aging hallmarks in the subject. In another embodiment, the present method ameliorates at least ten aging hallmarks in the subject. In another embodiment, the present method ameliorates at least eleven aging hallmarks in the subject.

For example, in one embodiment, the present method ameliorates at least the following aging hallmarks in the subject: genomic instability, an epigenetic alteration, and loss of proteostasis. In another embodiment, the present method ameliorates at least the following aging hallmarks in the subject: an epigenetic alteration, loss of proteostasis, and mitochondrial dysfunction. In another embodiment, the present method ameliorates at least the following aging hallmarks in the subject: an epigenetic alteration, loss of proteostasis, and deregulated nutrient sensing. In another embodiment, the present method ameliorates cellular senescence or reduces the amount of senescent cells. In another embodiment, the present method ameliorates cellular senescence and stem cell exhaustion. In another embodiment, the present method ameliorates mitochondrial dysfunction and loss of proteostasis. In another embodiment, the present method ameliorates epigenetic alterations and loss of proteostasis. In another embodiment, the present method ameliorates altered intercellular communication and deregulated nutrient sensing. In another embodiment, the present method ameliorates altered intercellular communication and stem cell exhaustion. In another embodiment, the present method ameliorates stem cell exhaustion and epigenetic alterations. In another embodiment, the present method ameliorates epigenetic alterations and mitochondrial dysfunction. In another embodiment, the present method ameliorates epigenetic alterations, loss of proteostasis, and mitochondrial dysfunction. In another embodiment, the present method ameliorates stem cell exhaustion, cellular senescence, and altered intercellular communication. In another embodiment, the present method ameliorates epigenetic alterations, cellular senescence, and mitochondrial dysfunction. In another embodiment, the present method ameliorates deregulated nutrient sensing, mitochondrial dysfunction, and loss or proteostasis. In another embodiment, the present method ameliorates epigenetic alterations, loss of proteostasis, mitochondrial dysfunction, and cellular senescence.

Also envisioned in this invention is administering to a subject the present composition in order to accomplish one or more of the following objectives: (i) increasing lifespan, lengthening healthspan, and/or improving stress resistance; (ii) slowing down aging; (iii) reducing, mitigating, and or slowing down aging-related diseases, aging-related symptoms, and/or age-related impairments; (iv) reducing the risk of, preventing, inhibiting the onset of, or treating a disease such as cardiovascular disease (e.g., atherosclerosis, high blood pressure, cardiac failure, cardiac hypertrophy, coronary syndrome, heart valve dysfunction, calcification of arteries and valves, or stroke), metabolic dysregulation (e.g., metabolic syndrome, high blood cholesterol, high serum triglycerides, increased serum HbA1c, insulin resistance, insulin resistance disorder, hyperglycemia, reduced insulin sensitivity, diabetes, obesity, a thyroid disorder, metabolic syndrome, fatty liver, steatohepatitis, hepatic steatosis, or diabetic retinopathy), cancer, cachexia, frailty, sarcopenia, neurodegenerative disease (e.g., Alzheimer's disease, vascular dementia, Lewy-body disease, frontotemporal dementia, Parkinson disease, mild cognitive impairment, amyotrophic lateral sclerosis, or Huntington's disease), neuropathy, eye disease (e.g., macular degeneration or cataract), lethargy, musculoskeletal diseases (e.g., rheumatoid arthritis, osteoarthritis, osteoporosis, or muscular dystrophy), lung diseases (e.g., lung fibrosis, chronic obstructive pulmonary disease (COPD), emphysema, aging-related increased susceptibility to lung infections including pneumonia), aging-related gastro-intestinal diseases (e.g., constipation, decreased stomach and gut motility, gastroparesis, polyps, gut dysbiosis, colitis, or inflammatory bowel disease), blood diseases (e.g., leukemia, lymphoma, anemia, platelet disorders, coagulation disorders, multiple myeloma, myelodysplastic syndromes, or myeloproliferative disorders), infertility, reduced fertility, aging-related increased risk of cancer or proliferative diseases, disorders caused or exacerbated by senescent cells, aging-related skin diseases, psoriasis, aging-related kidney diseases, sepsis, or sleep disorders; (v) improving and/or or mitigating the cellular and organismal physiological stress and dysfunction caused by surgery, exercise, combat, or certain work environments; and (vi) inhibiting the origin and progression of weight gain, wrinkles, sagging skin, aged skin, reduced stamina, reduced eyesight, reduced hearing, sarcopenia (i.e., a decrease in muscle mass), insulin resistance, fat deposition (e.g., abdominal fat deposition), hair graying, hair loss, baldness, loss of libido, erectile dysfunction, memory problems, reduced cognition, concentration problems, memory problems, sleep disorders, or mood disorders.

In a preferred embodiment of the present method, the subject is a human. In another embodiment of the present method, the subject is a rodent, a rabbit, a cat, a dog, or a horse.

Finally, this invention provides an article of manufacture comprising, in separate compartments, (i) the present composition, wherein the composition does not comprise butyrate or a butyrate equivalent, and (ii) butyrate and/or a butyrate equivalent. This invention also provides an article of manufacture comprising the present butyrate-or butyrate equivalent-comprising composition, wherein (i) the butyrate and/or butyrate equivalent and (ii) other components of the composition are in separate compartments.

In one embodiment of these articles, the butyrate and/or butyrate equivalent is the only agent separately (i.e., individually) encapsulated (e.g., in extended release form, while the other agents are in non-extended release form). In another embodiment, a plurality of agents is separately encapsulated (e.g., in extended release form). For example, the following embodiments are envisioned: (i) each of chondroitin, collagen peptide, butyrate, trehalose, and spermidine is separately encapsulated; (ii) each of chondroitin, collagen peptide, butyrate, and spermidine is separately encapsulated; (iii) each of chondroitin, collagen peptide, butyrate, and trehalose is separately encapsulated; (iv) each of chondroitin, collagen peptide, and butyrate is separately encapsulated; (v) each of butyrate, trehalose, and spermidine is separately encapsulated; (vi) each of butyrate and spermidine is separately encapsulated; and (vii) each of butyrate and trehalose is separately encapsulated.

One advantage of the present articles is the separation of foul-tasting butyrate and/or its foul-tasting equivalents from the other components of the present composition, thereby facilitating oral administration of the entire composition. In one example, the present article comprises (i) chondroitin, collagen peptide, trehalose, and spermidine (and, optionally, other agents as described herein) blended into a nutrient bar for oral consumption, and (ii) mini-capsules (or mini-spheres) of butyrate and/or a butyrate equivalent in the nutrient bar. In another example, the present article comprises (i) chondroitin, collagen peptide, trehalose, and spermidine (and, optionally, other agents as described herein) formulated as a powdered drink mix for oral consumption after combining with water, and (ii) one or more capsules of butyrate and/or a butyrate equivalent for oral consumption in conjunction with the drink. For instance, in one embodiment, the present article comprises, in separate compartments, (i) a composition (preferably in powder form) comprising 1,200 mg of chondroitin, 5,000 mg of collagen peptide, 10 g of trehalose, and 2 mg of spermidine (and optionally 500 mg of ascorbic acid, 1,200 mg of alpha-ketoglutarate, 1,500 mg of pomegranate or pomegranate equivalent, 1,200 mg of phosphatidylcholine (or phosphatidylserine), 200% of the U.S. recommended daily allowance of B vitamins, and 50 mg of apigenin), and (ii) 1,200 mg of butyrate or butyrate equivalent (preferably in capsule form). To reduce the foul smell and taste of butyrate, a triacylglyceride or diacylglyceride form of butyrate (such as tributyrin) can be used.

In the present method, it is envisioned that the present composition is orally administered to the subject daily. The following dosages are envisioned, without limitation: (i) 1,200 mg of chondroitin (or optionally, any of 10 mg, 20 mg, 30 mg, 40 mg, 50 mg, 60 mg, 70 mg, 80 mg, 90 mg, 100 mg, 150 mg, 200 mg, 300 mg, 400 mg, 500 mg, 600 mg, 700 mg, 900 mg, 1,000 mg, 1,100 mg, 1,200 mg, 1,300 mg, 1,400 mg, 1,500 mg, 1,600 mg, 2 g, 3 g, 4 g, 5 g, 10 g, and 20 g thereof); (ii) 5,000 mg of collagen peptide (or optionally, any of 10 mg, 20 mg, 50 mg, 100 mg, 200 mg, 300 mg, 400 mg, 500 mg, 1,000 mg, 1,500 mg, 2,000 mg, 2,500 mg, 3,500 mg, 4,000 mg, 5,000 mg, 6,000 mg, 8,000 mg, 10 g, 20 g, 30 g, 40 g, and 50 g thereof); (iii) 1,100 mg of butyrate (or optionally, any of 80 mg, 100 mg, 200 mg, 300 mg, 400 mg, 500 mg, 600 mg, 700 mg, 900 mg, 1,000 mg, 1,100 mg, 1,200 mg, 1,300 mg, 1,400 mg, 1,500 mg, and 1,600 mg thereof); (iv) 10 g of trehalose (or optionally, any of 0.5 g, 1 g, 2 g, 3 g, 4 g, 6 g, 7 g, 8 g, 9 g, 10 g, 15 g, 20 g, 25 g, and 30 g thereof); (v) 2 mg of spermidine (or optionally, any of 0.2 mg, 0.5 mg, 1 mg, 3 mg, 4 mg, 6 mg, 8 mg, 10 mg, 20 mg, 30 mg, 50 mg, 70 mg, 90 mg, and 110 mg thereof); (vi) 500 mg of ascorbic acid (or optionally, any of 10 mg, 20 mg, 30 mg, 40 mg, 50 mg, 60 mg, 70 mg, 80 mg, 90 mg, 120 mg, 140 mg, 160 mg, 180 mg, 200 mg, 400 mg, 500 mg, 1,000 mg, 1,500 mg, 2,000 mg, 3,000 mg, 4,000 mg, and 5,000 mg thereof); (vii) 1,100 mg of alpha-ketoglutarate (or optionally, any of 20 mg, 50 mg, 100 mg, 200 mg, 300 mg, 400 mg, 500 mg, 600 mg, 700 mg, 800 mg, 900 mg, 1,200 mg, 1,400 mg, 1,600 mg, 1,800 mg, 2,000 mg, 3,000 mg, 4,000 mg, 5,000 mg, 6,000 mg, 7,000 mg, 8,000 mg, 9,000 mg, and 10,000 mg thereof); (viii) 1,500 mg of pomegranate or pomegranate equivalent (or optionally, any of 150 mg, 300 mg, 500 mg, 1,000 mg, 2,000 mg, 3,000 mg, 4,000 mg, and 5,000 mg thereof); (ix) 1,200 mg of phosphatidylcholine (or optionally, any of 100 mg, 200 mg, 300 mg, 400 mg, 500 mg, 600 mg, 800 mg, 1,000 mg, 1,500 mg, 2,000 mg, and 3,000 mg thereof); (x) 200% of the U.S. recommended daily allowance of B vitamins; and (xi) 50 mg of apigenin (or optionally, any of 5 mg, 10 mg, 20 mg, 30 mg, 40 mg, 60 mg, 70 mg, 80 mg, 90 mg, 100 mg, 200 mg, 500 mg, 1,000 mg, and 2,000 mg thereof).

The following dosages are also envisioned for daily administration, without limitation: (i) 10 mg to 50 mg, 50 mg to 100 mg, 100 mg to 400 mg, 400 mg to 1,200 mg, 1,200 mg to 2 g, 2 g to 10 g, or 10 g to 20 g of chondroitin; (ii) 10 mg to 50 mg, 50 mg to 100 mg, 100 mg to 500 mg, 500 mg to 2,000 mg, 2,000 mg to 4,000 mg, 4,000 mg to 10 g, or 10 g to 50 g of collagen peptide; (iii) 80 mg to 200 mg, 200 mg to 400 mg, 400 mg to 1,200 mg, or 1,200 mg to 8,000 mg of butyrate; (iv) 0.5 g to 1 g, 1 g to 3 g, 3 g to 10 g, or 10 g to 50 g of trehalose; (v) 0.2 mg to 0.5 mg, 0.5 mg to 1 mg, 1 mg to 5 mg, or 5 mg to 30 mg of spermidine; (vi) 10 mg to 50 mg, 50 mg to 150 mg, or 150 mg to 5,000 mg of ascorbic acid; (vii) 100 mg to 400 mg, 400 mg to 800 mg, 800 mg to 1,200 mg, or 1,200 mg to 8,000 mg of alpha-ketoglutarate; (viii) 150 mg to 300 mg, 300 mg to 1,000 mg, or 1,000 mg to 10,000 mg of pomegranate or pomegranate equivalent; (ix) 100 mg to 400 mg, 400 mg to 800 mg, 800 mg to 1,500 mg, or 1,500 mg to 6,000 of phosphatidylcholine; (x) from 50% to 300% of the U.S. recommended daily allowance of B vitamins; and (xi) 5 mg to 10 mg, 10 mg to 30 mg, 30 mg to 70 mg, or 70 mg to 2,000 mg of apigenin.

The following three dosage regimens are (i) also envisioned for daily oral administration (in delayed release and/or extended release form, as well as non-delayed release, non-extended release form) and (ii) envisioned for the present compositions and articles of manufacture. They comprise, without limitation, the following: (i) 300 mg of chondroitin, 1,250 mg of collagen peptide, 275 mg of butyrate, 2.5 g of trehalose, 0.5 mg of spermidine, 125 mg of ascorbic acid, 275 mg of alpha-ketoglutarate, 375 mg of pomegranate or pomegranate equivalent, 300 mg of phosphatidylcholine, 50% of the U.S. recommended daily allowance of B vitamins, and 12.5 mg of apigenin; (ii) 600 mg of chondroitin, 2,500 mg of collagen peptide, 550 mg of butyrate, 5 g of trehalose, 1 mg of spermidine, 250 mg of ascorbic acid, 550 mg of alpha-ketoglutarate, 750 mg of pomegranate or pomegranate equivalent, 600 mg of phosphatidylcholine, 100% of the U.S. recommended daily allowance of B vitamins, and 25 mg of apigenin; and (iii) 900 mg of chondroitin, 3,750 mg of collagen peptide, 825 mg of butyrate, 7.5 g of trehalose, 1.5 mg of spermidine, 375 mg of ascorbic acid, 825 mg of alpha-ketoglutarate, 1,125 mg of pomegranate or pomegranate equivalent, 900 mg of phosphatidylcholine, 150% of the U.S. recommended daily allowance of B vitamins, and 37.5 mg of apigenin.

This invention will be better understood by reference to the examples which follow, but those skilled in the art will readily appreciate that the specific examples detailed are only illustrative of the invention as described more fully in the claims which follow thereafter.

### EXAMPLES

### Example 1—Additional Embodiments of the Present Compositions

The following additional combinations of agents are envisioned, without limitation, for the present compositions.

One additional combination comprises (i) chondroitin and (ii) butyrate or a butyrate equivalent. Another additional combination comprises (i) chondroitin and (ii) trehalose or a trehalose equivalent. A further additional combination comprises (i) chondroitin and (ii) spermidine or a spermidine equivalent. A further additional combination comprises (i) chondroitin, (ii) butyrate or a butyrate equivalent, and (iii) trehalose or a trehalose equivalent. A further additional combination comprises (i) chondroitin, (ii) butyrate or a butyrate equivalent, and (iii) spermidine or a spermidine equivalent. A further additional combination comprises (i) chondroitin, (ii) trehalose or a trehalose equivalent, and (iii) spermidine or a spermidine equivalent.

A further additional combination comprises (i) collagen peptide and (ii) butyrate or a butyrate equivalent. A further additional combination comprises (i) collagen peptide and (ii) trehalose or a trehalose equivalent. A further additional combination comprises (i) collagen peptide and (ii) spermidine or a spermidine equivalent. A further additional combination comprises (i) collagen peptide, (ii) butyrate or a butyrate equivalent, and (iii) trehalose or a trehalose equivalent. A further additional combination comprises (i) collagen peptide, (ii) butyrate or a butyrate equivalent, and (iii) spermidine or a spermidine equivalent. A further additional combination comprises (i) collagen peptide, (ii) trehalose or a trehalose equivalent, and (iii) spermidine or a spermidine equivalent.

A further additional combination comprises (i) chondroitin, (ii) collagen peptide and (iii) butyrate or a butyrate equivalent. A further additional combination comprises (i) chondroitin, (ii) collagen peptide and (iii) trehalose or a trehalose equivalent. A further additional combination comprises (i) chondroitin, (ii) collagen peptide and (iii) spermidine or a spermidine equivalent. A further additional combination comprises (i) chondroitin, (ii) collagen peptide, (iii) butyrate or a butyrate equivalent, and (iv) trehalose or a trehalose equivalent. A further additional combination comprises (i) chondroitin, (ii) collagen peptide, (iii) butyrate or a butyrate equivalent, and (iv) spermidine or a spermidine equivalent. A further additional combination comprises (i) chondroitin, (ii) collagen peptide, (iii) trehalose or a trehalose equivalent, and (iv) spermidine or a spermidine equivalent.

A further additional combination comprises chondroitin, collagen peptide, butyrate, trehalose, spermidine, and ascorbic acid. A further additional combination comprises chondroitin, collagen peptide, butyrate, trehalose, spermidine, and alpha-ketoglutarate. A further additional combination comprises chondroitin, collagen peptide, butyrate, trehalose, spermidine, and pomegranate or a pomegranate equivalent. A further additional combination comprises chondroitin, collagen peptide, butyrate, trehalose, spermidine, and phosphatidylcholine. A further additional combination comprises chondroitin, collagen peptide, butyrate, trehalose, spermidine, and a B vitamin. A further additional combination comprises chondroitin, collagen peptide, butyrate, trehalose, spermidine, and apigenin. A further additional combination comprises chondroitin, collagen peptide, butyrate, trehalose, spermidine, and ascorbic acid, alpha-ketoglutarate, pomegranate or a pomegranate equivalent, phosphatidylcholine, a B vitamin, and apigenin.

A further additional combination comprises a histone deacetylase inhibitor, a polyamine, a proteostasis-supporting saccharide, a chondroitin-based substance, collagen peptide, a ascorbic acid derivative, alpha ketoglutarate, pomegranate derivatives, a phospholipid, B vitamins, and apigenin.

A further additional combination comprises a histone deacetylase inhibitor, a polyamine, a proteostasis-supporting saccharide, a chondroitin-based substance, collagen peptide, a ascorbic acid derivative, alpha ketoglutarate, a phospholipid, B vitamins, and apigenin.

A further additional combination comprises a histone deacetylase inhibitor, a polyamine, a proteostasis-supporting saccharide, a chondroitin-based substance, collagen peptide, a ascorbic acid derivative, alpha ketoglutarate, a phospholipid, and B vitamins.

A further additional combination comprises a histone deacetylase inhibitor, a polyamine, a proteostasis-supporting saccharide, a chondroitin-based substance, collagen peptide, a ascorbic acid derivative, and alpha ketoglutarate.

A further additional combination comprises a histone deacetylase inhibitor, a polyamine, a proteostasis-supporting saccharide, a chondroitin-based substance, and collagen peptide.

A further additional combination comprises a histone deacetylase inhibitor, a polyamine, a proteostasis-supporting saccharide, and a chondroitin-based substance.

A further additional combination comprises a histone deacetylase inhibitor, spermidine, trehalose, chondroitin, collagen peptides, a ascorbic acid derivative, alpha ketoglutarate, ellagic acid, a phospholipid, B vitamins, and apigenin or an apigenin-derivative.

A further additional combination comprises a histone deacetylase inhibitor, spermidine, trehalose, chondroitin, collagen peptides, a ascorbic acid derivative, a phospholipid, B vitamins, and apigenin.

A further additional combination comprises a histone deacetylase inhibitor, spermidine, chondroitin, collagen peptides, a ascorbic acid derivative, a phospholipid, and B vitamins.

A further additional combination comprises butyrate, spermidine, chondroitin sulphate, collagen peptides, a ascorbic acid derivative, alpha-ketoglutarate, phosphatidylcholine, and B vitamins.

A further additional combination comprises butyrate, spermidine, chondroitin sulphate, collagen peptides, ascorbic acid, alpha-ketoglutarate, phosphatidylcholine, and B vitamins.

A further additional combination comprises butyrate, spermidine, trehalose, chondroitin sulphate, collagen peptides, a ascorbic acid derivative, and phosphatidylcholine.

A further additional combination comprises trehalose, spermidine, chondroitin sulphate, collagen peptides, a ascorbic acid derivative, alpha-ketoglutarate, phosphatidylcholine, and B vitamins.

A further additional combination comprises butyrate, spermidine, chondroitin sulphate, collagen peptides, a ascorbic acid derivative, alpha-ketoglutarate, phosphatidylcholine, and B vitamins.

A further additional combination comprises spermidine, chondroitin sulphate, collagen peptides, a ascorbic acid derivative, alpha-ketoglutarate, phosphatidylcholine, and B vitamins.

A further additional combination comprises a supplement or nutraceutical comprising a histone deacetylase inhibitor such as for example but not limited to butyrate, spermidine, trehalose, chondroitin, collagen peptides, a ascorbic acid derivative, alpha ketoglutarate, ellagic acid, a phospholipid, B vitamins, and apigenin or an apigenin-derivative.

A further additional combination comprises a food or snack comprising a histone deacetylase inhibitor (such as butyrate), spermidine, trehalose, chondroitin, collagen peptides, a ascorbic acid derivative, alpha ketoglutarate, ellagic acid, a phospholipid, B vitamins, and apigenin or an apigenin-derivative.

A further additional combination comprises a food or snack comprising a histone deacetylase inhibitor (such as butyrate), spermidine, trehalose, chondroitin, collagen peptides, a ascorbic acid derivative, alpha ketoglutarate, ellagic acid, a phospholipid, B vitamins, apigenin or an apigenin-derivative, and either all required macronutrients (e.g., fats, carbohydrates, and protein) or specific groups of macronutrients (e.g., fats, carbohydrates, or protein).

A further additional combination comprises a food or snack comprising spermidine, trehalose, chondroitin, collagen peptides, a ascorbic acid derivative, alpha ketoglutarate, pomegranate extract, a phospholipid, B vitamins, and apigenin.

A further additional combination comprises a food or snack comprising spermidine, trehalose, chondroitin and collagen peptides.

A further additional combination comprises a skin cream, skin serum, or skincare product comprising a histone deacetylase inhibitor (such as butyric acid), spermidine, trehalose, chondroitin, collagen peptides, a ascorbic acid derivative, alpha ketoglutarate, ellagic acid, a phospholipid, B vitamins, and apigenin or an apigenin-derivative.

In yet an additional embodiment of the present method, the daily dose administered comprises (i) 40 mg to 10,000 mg of butyrate (ideally 400 mg to 4,000 mg), (ii) 0.1 mg to 20 mg of spermidine (average 1 mg to 6 mg of spermidine), (iii) 0.2 g to 70 g of trehalose, (iv) 20 mg to 4,000 mg of chondroitin, (v) 20 mg to 15,000 mg of collagen peptides, (vi) 10 to 5,000 mg of ascorbic acid, (vii) 20 mg to 10,000 mg of alpha-ketoglutarate (average around 1, 100 mg), (viii) 10 mg to 10,000 mg of pomegranate or pomegranate derivative or pomegranate extract containing 50 mg to 1,000 mg or urolithin or pomegranate extract standardised to 30% punicalagins or pomegranate blend standardised to 20% punicic acid or pomegranate extract standardised to contain 40% ellagic acid, (ix) 20 mg to 4,000 mg of phosphatidylcholine (average ideal dose 1,200 mg), (x) 100% to 300% or the U.S. recommended daily allowances of B vitamins, and (xi) 50 mg of apigenin.

### Example 2—Measuring Epigenetic Dysregulation

The success of the present composition in ameliorating epigenetic dysregulation in a subject (e.g., a human subject) can be measured, for example, by making any of the following determinations: (i) the subject's epigenetic methylation, histonylation, and/or chromatin patterns more closely resemble the patterns of younger persons (e.g., by at least 1%, at least 2%, at least 5%, at least 10%, at least 20%, at least 30%, at least 40%, at least 50%, at least 60%, at least 70%, at least 80%, at least 90%, or at least 100%); (ii) an epigenetic clock (e.g., a Horvath clock or a Levine clock)-measuring methylation of DNA in different regions of the subject's DNA more closely resembles the DNA methylation pattern of a younger subject (e.g., by at least 1%, at least 2%, at least 5%, at least 10%, at least 20%, at least 30%, at least 40%, at least 50%, at least 60%, at least 70%, at least 80%, at least 90%, or at least 100%); (iii) an epigenetic DNA methylation clock (e.g., a Horvath clock or a Levine clock) shows that an adult human subject has been rejuvenated by at least 3 months, one year, at least two years, or at least three years after being administered the present composition (or of which the epigenetic age or biological age has been decreased by at least 1%, at least 2%, at least 3%, at least 5%, at least 10%, at least 20%, at least 30%, at least 40%, at least 50%, at least 60%, at least 70%, at least 80%, at least 90%, or at least 100% compared to a subject's chronological age); (iv) the subject's chromatin distribution more closely resembles that of a younger subject (e.g., by at least 0.2%, at least 1%, at least 2%, at least 5%, at least 10%, at least 20%, at least 30%, at least 40%, at least 50%, at least 60%, at least 70%, at least 80%, at least 90%, or at least 100%); (v) the subject's transcriptome resembles the transcriptome of a younger subject (e.g., by at least 0.2%, at least 0.5%, at least 1%, at least 2%, at least 5%, at least 10%, at least 20%, at least 30%, at least 40%, at least 50%, at least 60%, at least 70%, at least 80%, at least 90%, or at least 100%); (vi) the subject's expression profile of ribosome-related proteins and/or ribosome-related RNA more closely resembles that of a younger organism (e.g., by at least 0.2%, at least 0.5%, at least 1%, at least 2%, at least 5%, at least 10%, at least 20%, at least 30%, at least 40%, at least 50%, at least 60%, at least 70%, at least 80%, at least 90%, or at least 100%); or (vii) the subject's levels of sirtuins have increased (e.g., by at least 1%, at least 2%, at least 5%, at least 10%, at least 20%, at least 30%, at least 40%, at least 50%, at least 60%, at least 70%, at least 80%, at least 90%, or at least 100%).

The success of the present composition in ameliorating epigenetic dysregulation in a subject (e.g., a human subject) can also be measured, for example, by making any of the following determinations: (i) an increase in the level of H3K9me3 in the subject (e.g., by at least 1%, at least 2%, at least 5%, at least 10%, at least 20%, at least 30%, at least 40%, at least 50%, at least 60%, at least 70%, at least 80%, at least 90%, or at least 100%); or (ii) an increase in the level of heterochromatin protein (Hp1y) in the subject (e.g., by at least 1%, at least 2%, at least 5%, at least 10%, at least 20%, at least 30%, at least 40%, at least 50%, at least 60%, at least 70%, at least 80%, at least 90%, or at least 100%).

### Example 3—Measuring Cell Senescence

The success of the present composition in ameliorating cell senescence in a subject (e.g., a human subject) can be measured, for example, by making any of the following determinations: (i) a delay in the occurrence of senescent cells or a delay in the transition of normal cells into senescent cells in vitro or in vivo (e.g., by at least 2%, at least 5%, at least 10%, at least 20%, at least 30%, at least 40%, at least 50%, at least 60%, at least 70%, at least 80%, at least 90%, or at least 100%); and/or (ii) a decrease in the number of senescent cells in vitro or in vivo (e.g., by at least 1%, at least 2%, at least 5%, at least 10%, at least 20%, at least 30%, at least 40%, at least 50%, at least 60%, at least 70%, at least 80%, at least 90%, or at least 100%). Senescence or senescent-like status of cells can be measured, for example, via one or more of the following: (i) epigenetic profiling such as measuring epigenetic markers of senescence (e.g., changes in histone H3 lysine 9 and 27 trimethylation (changed levels of H3K9me3 and H3K27me3), changed heterochromatin protein 1 (HP1 ) family protein levels, increased histone variant macroH2A levels, and/or chromatin remodeling enzyme ATRX levels); (ii) changes in cell morphology resembling the morphology of senescent cells, (e.g., enlarged size, a more flattened shape, polyploid nuclei, or accumulation of DNA damage foci); (iii) changes in levels of nuclear lamina-associated proteins such as lamins (e.g., a decline lamin B1); (iv) heterochromatin changes more resembling the heterochromatin status of senescent cells (e.g., an increase in senescence-associated heterochromatic foci (SAHF)); (v) an increase of senescence-associated distention of satellites (SADS) or other changes in pericentric satellite DNA more resembling that of senescent cells; and (vi) changes in secretory phenotype corresponding to that of senescent cells (e.g., the occurrence of a more senescence-associated secretome including, without limitation, cytokines, interleukins (e.g., IL-1, IL-2, IL-6, II-8, and TNF-alpha), matrix metalloproteinases, pro angiogenetic factors, pro-inflammatory substances, and growth factors (e.g., vascular endothelial growth factor, insulin like growth factor, and GM-CSF)).

The success of the present composition in ameliorating cell senescence in a subject (e.g., a human subject) can also be measured, for example, by making any of the following determinations: (i) an increase in the level of H3K9me3 in the subject (e.g., by at least 5%, at least 10%, at least 20%, at least 30%, at least 40%, at least 50%, at least 60%, at least 70%, at least 80%, at least 90%, or at least 100%); (ii) an increase in the level of H3K27me3 in the subject (e.g., by at least 5%, at least 10%, at least 20%, at least 30%, at least 40%, at least 50%, at least 60%, at least 70%, at least 80%, at least 90%, or at least 100%); (iii) a decrease in the level of P16INK4A in the subject (e.g., by at least 5%, at least 10%, at least 20%, at least 30%, at least 40%, at least 50%, at least 60%, at least 70%, at least 80%, at least 90%, or at least 100%); or (iv) a decrease in the level of one or more of p16, p53, p21, p14, p15, and beta-galactosidase activity in the subject (e.g., by at least 5%, at least 10%, at least 20%, at least 30%, at least 40%, at least 50%, at least 60%, at least 70%, at least 80%, at least 90%, or at least 100%).

### Example 4—Measuring Altered Intercellular Communication

The success of the present composition in ameliorating altered intercellular communication in a subject (e.g., a human subject) can be measured, for example, by determining a decrease in the level of one or more of IL-1, IL-2, IL-6, IL-8, IL-17a, and TNF-alpha signaling in the subject (e.g., by at least 5%, at least 10%, at least 20%, at least 30%, at least 40%, at least 50%, at least 60%, at least 70%, at least 80%, at least 90%, or at least 100%).

### Example 5—Measuring Autophagy

The success of the present composition in ameliorating decreased autophagy in a subject (e.g., a human subject) can be measured, for example, by determining any of the following: (i) an increase in the level of one or more of LC3, p62, Ulk1, Parkin, PINK1, LAMP2A, Atg, FIP200, Vps15, Beclin, Lamp-1, Lamp-2, Hsp70, Hsp90, and SQSTM1 in the subject (e.g., by at least 5%, at least 10%, at least 20%, at least 30%, at least 40%, at least 50%, at least 60%, at least 70%, at least 80%, at least 90%, or at least 100%); (ii) an increase (e.g., by at least 5%, at least 10%, at least 20%, at least 30%, at least 40%, at least 50%, at least 60%, at least 70%, at least 80%, at least 90%, or at least 100%) in any of phagophore formation, autophagosome formation, lysosome numbers, lysosomal activity, LC3 puncta, lysosomal content (as determined, for example, using immunochemistry, electron microscopy, Western blotting, or flow cytometry); (iii) increased lysosome acidity (e.g., by at least 5%, at least 10%, at least 20%, at least 30%, at least 40%, at least 50%, at least 60%, at least 70%, at least 80%, at least 90%, or at least 100%); (iv) upregulation of the ubiquitin-proteasome system (e.g., by at least 5%, at least 10%, at least 20%, at least 30%, at least 40%, at least 50%, at least 60%, at least 70%, at least 80%, at least 90%, or at least 100%); or (v) inhibition of mTOR or the mTOR pathway (e.g., by at least 5%, at least 10%, at least 20%, at least 30%, at least 40%, at least 50%, at least 60%, at least 70%, at least 80%, at least 90%, or at least 100%).

### Example 6—Measuring Cell Oxidation

The success of the present composition in ameliorating cell oxidation in a subject (e.g., a human subject) can be measured, for example, by making any of the following determinations: (i) a decrease in the level of 8-isoprostane in the subject (e.g., by at least 5%, at least 10%, at least 20%, at least 30%, at least 40%, at least 50%, at least 60%, at least 70%, at least 80%, at least 90%, or at least 100%); (ii) a decrease in the level of 8-hydroxydeoxyguanosine (8-OHdG) in the subject (e.g., by at least 5%, at least 10%, at least 20%, at least 30%, at least 40%, at least 50%, at least 60%, at least 70%, at least 80%, at least 90%, or at least 100%); or (iii) a decrease in the subject of levels of any of oxidized LDL, malondialdehyde (MDA), 4-HNA, lipid hydroperoxides, oxidized proteins, protein carbonylation and protein nitration (3-nitrotyrosines), advanced glycation end products (AGEs), and advanced oxidation protein products (e.g., by at least 5%, at least 10%, at least 20%, at least 30%, at least 40%, at least 50%, at least 60%, at least 70%, at least 80%, at least 90%, or at least 100%).

### Example 7—Measuring Telomere Fitness

The success of the present composition in ameliorating telomere attrition in a subject (e.g., a human subject) can be measured, for example, by making any of the following determinations: (i) an increase in telomere length in the subject (e.g., by at least 0.01%, at least 0.1%, at least 0.5%, at least 1.0%, at least 5%, at least 10%, at least 20%, at least 30%, at least 40%, or at least 50%); or (ii) an increase in mRNA expression of telomerase, TERT, TERC, or telomerase-related genes (e.g., by at least 1%, at least 2%, at least 5%, at least 10%, at least 20%, at least 30%, at least 40%, at least 50%, at least 60%, at least 70%, at least 80%, at least 90%, or at least 100%).

### Example 8—Measuring Stem Cell Fitness

The success of the present composition in ameliorating stem cell exhaustion in a subject (e.g., a human subject) can be measured, for example, by determining an increase in the number of stem cells in vivo (e.g., by at least 1%, at least 2%, at least 5%). This increase can be measured in vitro or in vivo via, for example, one or more of the following biomarkers (i) muscle satellite stem cell biomarkers (e.g., PAX7, LMNA, and emerin); (ii) neuronal stem cell biomarkers (e.g., Nestin, Sox2, ASCL1/Mash1, ABCG2, BMI-1, beta-Catenin, Brg1, N-Cadherin, Calcitonin R, CD15/Lewis X, CD133, CDCP1, COUP-TF I/NR2F1, CXCR4, FABP7/B-FABP, FABP8/M-FABP, FGFR2, FGFR4, FoxD3, Frizzled-9, GCNF/NR6A1, GFAP, Glut1, HOXB1, ID2, LRTM1, Meteorin, MSX1, Musashi-1, Musashi-2, Nestin, NeuroD1, Noggin, Notch-1, Notch-2, Nrf2, Nucleostemin, Numb, Otx2, Pax3, Pax6, PDGF R alpha, PKC zeta, Prominin 2, ROR2, RUNX1/CBFA2, RXR alpha/NR2B1 sFRP-2, SLAIN1, SOX1, SOX2, SOX9, SOX11, SOX21, SSEA-1, SSEA-4, TRAF-4, Vimentin, and ZIC1); (iii) pluripotency stem cell markers (e.g., Oct4, NANOG, Sox2, and Myc); (iv) hematopoietic stem cell markers (e.g., CD34, CD59, and CD90/Thy1); and (v) mesenchymal stem cell markers (e.g., CD105, CD90, CD73, CD44, CD45, CD29, CD166, Stro-1, CD106, and GSTT1).

Since measuring stem cells in vivo is difficult and costly, success of the present composition in ameliorating stem cell exhaustion can also be measured, for example, by making the following preferred in vitro (“lab dish”) determinations (as well as in vivo determinations): (i) an increase in the number of stem cells (e.g., by at least 2%, at least 5%, at least 10%, at least 20%, at least 30%, at least 40%, at least 50%, at least 60%, at least 70%, at least 80%, at least 90%, or at least 100%); (ii) increased viability of the stem cells (e.g., by at least 2%, at least 5%, at least 10%, at least 20%, at least 30%, at least 40%, at least 50%, at least 60%, at least 70%, at least 80%, at least 90%, or at least 100%); (iii) an increase in potency of the stem cells in vitro (and in vivo as well) (e.g., by at least 2%, at least 5%, at least 10%, at least 20%, at least 30%, at least 40%, at least 50%, at least 60%, at least 70%, at least 80%, at least 90%, or at least 100%); (iv) an increase in the quality of stem cells, measured for example by an increase in growth of the stem cells, an increase in the ability to proliferate, an increase in the ability to form colonies, or an increase in the ability to produce cells (e.g., by at least 2%, at least 5%, at least 10%, at least 20%, at least 30%, at least 40%, at least 50%, at least 60%, at least 70%, at least 80%, at least 90%, or at least 100%); (v) an increased lifespan of stem cells, as measured in hours (e.g., by at least 2%, at least 5%, at least 10%, at least 20%, at least 30%, at least 40%, at least 50%, at least 60%, at least 70%, at least 80%, at least 90%, or at least 100%); or (vi) an increased resistance of stem cells against cellular stressors (e.g., heat, cold, or toxins) (e.g., by at least 2%, at least 5%, at least 10%, at least 20%, at least 30%, at least 40%, at least 50%, at least 60%, at least 70%, at least 80%, at least 90%, or at least 100%).

The success of the present composition in ameliorating stem cell exhaustion in a subject (e.g., a human subject) can further be measured, for example, by making any of the following determinations in vitro, but also in vivo: (i) improved stem cell function as measured, for example, by replication ability, potency, proliferation capability, survival (as measured, for example, temporally (e.g., number of extra hours of survival), by exposing stem cells to physiological stressors and toxins), potency, and quality (e.g., stem cells have an increased number of replications or can generate/produce larger numbers of cells stemming from the stem cells or display increase cell division markers (e.g., cyclin D1 or increased B-catechin) (e.g., by at least 5%, at least 10%, at least 20%, at least 30%, at least 40%, at least 50%, at least 60%, at least 70%, at least 80%, at least 90%, or at least 100%); (ii) improved stem cell transcriptome (i.e., the stem cell's transcriptome is more similar to the transcriptome of a younger stem cell or a more functional stem cell) (e.g., by at least 0.5%, at least 1%, at least 2%, at least 5%, at least 10%, at least 20%, at least 30%, at least 40%, at least 50%, at least 60%, at least 70%, at least 80%, at least 90%, or at least 100%); (iii) improved stem cell proteome (i.e., the stem cell's proteome is more similar to the proteome of a younger stem cell or more functional stem cell) (e.g., by at least 0.5%, at least 1%, at least 2%, at least 5%, at least 10%, at least 20%, at least 30%, at least 40%, at least 50%, at least 60%, at least 70%, at least 80%, at least 90%, or at least 100%); or (iv) an improved stem cell epigenome (i.e., the stem cell's epigenome is more similar to the epigenome of a younger stem cell or more functional stem cell) (e.g., by at least 0.5%, at least 1%, at least 2%, at least 5%, at least 10%, at least 20%, at least 30%, at least 40%, at least 50%, at least 60%, at least 70%, at least 80%, at least 90%, or at least 100%), as assessed, for example, by measuring methylation or histonylation patterns and comparing them to those of younger, more functional, or more potent stem cells.

### Example 9—Measuring Physiological Biomarkers as Indicators of Stem Cell Fitness and Senescent Cell Levels in an Organism

The success of the present composition in ameliorating adverse physiological events in a subject (e.g., a human subject) can be measured, for example, by making any of the following determinations: (i) a decrease in the number and/or severity of skin wrinkles as measured, for example, via wrinkle surface area (e.g., via 3D topography or profilometry); (ii) an increase in skin radiance as measured, for example, via an identometer, cutometer, transepidermal water loss meter, corneometer, or a skin transcriptome or proteome biomarker panel that shows improved skin functioning; (iii) improved cognition as measured, for example, via a cognitive test; (iv) improved feelings of happiness as measured, for example, via the Depressive Symptoms Scale; (v) reduced frailty, as measured, for example, via an international frailty scale or mobility scale (such as the Canadian Study on Health & Aging Clinical Frailty Scale (CSHA-CFS)); (vi) improved reaction time; (vii) improved neuromuscular capability and strength (e.g., increased stamina, improved balance, improved proprioception, improved mobility (measured by 6-minute walk test (6MWT), 4-m gait speed test (4MGST), improved short physical performance battery (SPPB) score, or tests measuring balance, gait speed tests, chair stand tests or other markers of physical function), increased grip strength, increased power, increased activity levels, improved 400 m walk test, and improved arm curl test results); (viii) improved blood flow, as measured by increase of unit of blood per mm³ of tissue; (ix) reduced blood pressure; (x) improved vascular health, as measured, for example, via pulse wave velocity of the blood vessels or flow-mediated vasodilatation; (xi) induced weight loss, a reduction in waist circumference or BMI, or a reduction in abdominal fat volume; (xii) improvement in heart rate variability (HRV); and (xiii) improvement in an AI-assessed facial aging profile (e.g., as measured via facial AI software that for example assesses wrinkle area, blood flow, or skin sagging).

Importantly, if stem cell health is improved and cell senescence is reduced, the various physiological biomarkers discussed in this example will also improve. This is particularly the case with stem cells in that reducing the aging hallmark of stem cell decline (the measurement of which is discussed above) improves physiological biomarkers.

### Example 10—Measuring Genomic Fitness

The success of the present composition in ameliorating genomic instability in a subject (e.g., a human subject) can be measured, for example, by determining a decrease in the level of 8-hydroxydeoxyguanosine (8-OHdG), gamma-H2AX, or biomarkers of DNA mutations (e.g., double strand breaks) in the subject (e.g., by at least 5%, at least 10%, at least 20%, at least 30%, at least 40%, at least 50%, at least 60%, at least 70%, at least 80%, at least 90%, or at least 100%).

### Example 11—Measuring Improvement in Deregulated Nutrient Sensing

The success of the present composition in ameliorating deregulated nutrient sensing in a subject (e.g., a human subject) can be measured, for example, by making any of the following determinations: (i) an increase in the level of insulin sensitivity in the subject (e.g., by at least 5%, at least 10%, at least 20%, at least 30%, at least 40%, at least 50%, at least 60%, at least 70%, at least 80%, at least 90%, or at least 100%); or (ii) a decrease in the level of fasting glucose, fasting insulin, LDL, HbA1c, mTOR activity, or mitochondrial reactive oxygen species in the subject (e.g., by at least 5%, at least 10%, at least 20%, at least 30%, at least 40%, at least 50%, at least 60%, at least 70%, at least 80%, at least 90%, or at least 100%).

### REFERENCES

1. D. Harman, The Aging Process. Proc. Nat'l. Acad. Sci. U.S.A., 1981 Nov; 78(11):7124-8.
2. G. Bjelakovic, et al., Mortality in randomized trials of antioxidant supplements for primary and secondary prevention: systematic review and meta-analysis. JAMA, 2007, Feb 28; 297(8):842-57.
3. H. Macpherson, et al., Multivitamin-multimineral supplementation and mortality: a meta-analysis of randomized controlled trials. Am. J. Clin. Nutr., 2013, Feb; 97(2):437-44.
4. H.D. Sesso, et al., Multivitamins in the prevention of cardiovascular disease in men: the Physicians' Health Study II randomized controlled trial. JAMA, 2012, Nov 7; 308(17):1751-60.
5. W. Yang and S. Hekimi, A Mitochondrial Superoxide Signal Triggers Increased Longevity in Caenorhabditis elegans. PLOS Biology, 2010 Dec 7; 8(12):e1000556.
6. M. Ristow, et al., Antioxidants prevent health-promoting effects of physical exercise in humans. Proc. Nat'l. Acad. Sci. U.S.A., 2009, 106, 8665-8670.
7. C. López-Otín, et al., the Hallmarks of Aging, Cell, Vol. 153, Issue 6, pp. 1194-1217 (2013).
8. W. Zhang, et al., The ageing epigenome and its rejuvenation. Nature Reviews Mol. Cell Biol., 21, pages 137-150 (2020).
9. P. Sen, et al., Epigenetic Mechanisms of Longevity and Aging, Cell, 2016, Aug 11; 166(4): 822-839.

1. A composition comprising (i) chondroitin and/or collagen peptide and (ii) at least one agent selected from the group consisting of butyrate, a butyrate equivalent, trehalose, a trehalose equivalent, spermidine, and a spermidine equivalent.

2. The composition of claim 1, wherein the composition comprises (i) chondroitin and (ii) at least one agent selected from the group consisting of butyrate, a butyrate equivalent, trehalose, a trehalose equivalent, spermidine, and a spermidine equivalent.

3. The composition of claim 2, wherein the composition comprises chondroitin, butyrate, trehalose, and spermidine.

4. The composition of claim 1, wherein the composition comprises (i) collagen peptide and (ii) at least one agent selected from the group consisting of butyrate, a butyrate equivalent, trehalose, a trehalose equivalent, spermidine, and a spermidine equivalent.

5. The composition of claim 4, wherein the composition comprises collagen peptide, butyrate, trehalose, and spermidine.

6. The composition of claim 1, wherein the composition comprises (i) chondroitin, (ii) collagen peptide, and (iii) at least one agent selected from the group consisting of butyrate, a butyrate equivalent, trehalose, a trehalose equivalent, spermidine, and a spermidine equivalent.

7. The composition of claim 1, wherein the trehalose equivalent is mannitol, glucosamine, acetyl-glucosamine, galactosamine, mannosamine, glycerol, erythritol, or xylitol.

8. The composition of claim 1, wherein the spermidine equivalent is putrescine, spermine, norspermidine, acetyl spermidine, diacetyl spermine, methylated spermidine, a methylated polyamine, or a spermidine salt.

9. The composition of claim 1, wherein the butyrate equivalent is acetic acid.

10. The composition of claim 6, wherein the composition comprises chondroitin, collagen peptide, butyrate, trehalose, and spermidine.

11. The composition of claim 10, wherein the composition comprises 50 mg-6,000 mg of chondroitin, 50 mg-20,000 mg of collagen peptide, 50 mg-12,000 mg of butyrate, 0.1 g-50 g of trehalose, and 0.1 mg-30 mg of spermidine.

12. The composition of claim 11, wherein the composition comprises 1,200 mg of chondroitin, 5,000 mg of collagen peptide, 1,100 mg of butyrate, 10 g of trehalose, and 2 mg of spermidine.

13. The composition of claim 10, wherein the composition comprises (i) chondroitin, collagen peptide, butyrate, trehalose, and spermidine, and (ii) one or more agents selected from the group consisting of ascorbic acid or an ascorbic acid equivalent, alpha-ketoglutarate or an alpha-ketoglutarate equivalent, pomegranate or a pomegranate equivalent, a phospholipid, a B vitamin, and apigenin or an apigenin equivalent.

14. The composition of claim 13, wherein the composition comprises chondroitin, collagen peptide, butyrate, trehalose, spermidine, ascorbic acid, and alpha ketoglutarate.

15. The composition of claim 13, wherein the composition comprises chondroitin, collagen peptide, butyrate, trehalose, spermidine, ascorbic acid, alpha ketoglutarate, and phosphatidylcholine.

16. The composition of claim 10, wherein the composition comprises chondroitin, collagen peptide, butyrate, trehalose, spermidine, ascorbic acid, alpha ketoglutarate, phosphatidylcholine, and a B vitamin.

17. The composition of claim 10, wherein the composition comprises chondroitin, collagen peptide, butyrate, trehalose, spermidine, ascorbic acid, alpha ketoglutarate, phosphatidylcholine, a B vitamin, and apigenin or an apigenin equivalent.

18. The composition of claim 13, wherein the composition comprises 400 mg-1,200 mg of chondroitin, 1,000 mg-5,000 mg of collagen peptide, 400 mg-1,200 mg of butyrate, 1 g-20 g of trehalose, 0.2 mg-20 mg of spermidine, 500 mg of ascorbic acid, 500 mg-4,000 mg of alpha-ketoglutarate, 100 mg to 3,000 mg of pomegranate or pomegranate equivalent, 250 mg-2,400 mg of phosphatidylcholine, 100%-300% of the U.S. recommended daily allowance of B vitamins, and 50 mg of apigenin.

19. The composition of claim 18, wherein the composition comprises 1,200 mg of chondroitin, 5,000 mg of collagen peptide, 1,100 mg of butyrate, 10 g of trehalose, 2 mg of spermidine, 500 mg of ascorbic acid, 1,100 mg of alpha-ketoglutarate, 1,500 mg of pomegranate or pomegranate equivalent, 1,200 mg of phosphatidylcholine, 200% of the U.S. recommended daily allowance of B vitamins, and 50 mg of apigenin.

20. The composition of claim 1, wherein one or more of the agents in the composition is formulated for delayed release and/or extended release.

21. A composition comprising (i) the composition of claim 1 and (ii) a pharmaceutically acceptable carrier.

22. The composition of claim 1, wherein the composition is in a form selected from the group consisting of a food, a nutritional supplement, a nutraceutical, and a skincare product.

23. A method for ameliorating at least one aging hallmark in a subject comprising administering to the subject the composition of claim 1, wherein the aging hallmark is selected from the group consisting of genomic instability, telomere attrition, an epigenetic alteration, loss of proteostasis, deregulated nutrient sensing, mitochondrial dysfunction, cellular senescence, stem cell exhaustion, transcriptomic dysregulation, crosslinking, and altered intercellular communication.

24. The method of claim 23, wherein the method ameliorates genomic instability, loss of proteostasis, one or more epigenetic alterations, telomere attrition, deregulated nutrient sensing, mitochondrial dysfunction, cellular senescence, stem cell exhaustion, transcriptomic dysregulation, crosslinking, and altered intercellular communication.

25. The method of claim 23, wherein the subject is a human.

26. The method of claim 23, wherein the subject is a rodent, a rabbit, a cat, a dog, or a horse.

27. An article of manufacture comprising, in separate compartments, (i) the composition of claim 1, wherein the composition does not comprise butyrate or a butyrate equivalent, and (ii) butyrate and/or a butyrate equivalent.

28. An article of manufacture comprising the composition of claim 1, wherein (i) the butyrate and/or butyrate equivalent and (ii) other components of the composition are in separate compartments.
